(19) United States
(12) Patent Application Publication
(10) Pub. No.: US 2008/0119759 A1
(43) Pub. Date: May 22, 2008

(54) METHOD AND APPARATUS FOR ASPIRATING BONE MARROW

(75) Inventor: Robert F. McLain, Pepper Pike, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/938,386

(22) Filed: Nov. 12, 2007

Related U.S. Application Data
(60) Provisional application No. 60/860,411, filed on Nov. 21, 2006.

(51) Int. Cl. A61B 10/00; A61B 17/56; A61B 17/58

(52) U.S. Cl. 600/567; 606/104; 606/73

(57) ABSTRACT

An apparatus and method for use in aspirating bone marrow is provided. The apparatus comprises a tube which has a body with a distal end portion. The tube has a central opening at one end of a central passage extending through the tube and through which bone marrow is aspirated. The tube is also tapered. A thread convolution on the body of the tube at the distal end portion of the tube is also provided as an alternate design. At least one aspiration opening between the central opening and the thread convolution is used to further aspirate bone marrow.

### RELATED PATENT APPLICATION

This application claims priority from U.S. Provisional Patent application Ser. No. 60/860,411, filed Nov. 21, 2006, the subject matter of which is incorporated herein by reference.

### TECHNICAL FIELD

The present invention is directed to aspirating bone marrow and, in particular, is directed to a method and apparatus for aspirating bone marrow through a human vertebra.

### BACKGROUND OF THE INVENTION

Segmental pedicle screw fixation is now the most widely used method of surgical fixation of the spinal column. Studies have shown that, when done correctly, pedicle screw instrumentation introduces no additional risk to spinal surgical procedures. However, to obtain the benefits of pedicle screw fixation without increasing the risk to the patient, the surgeon must have specific and hands-on training in pedicle screw techniques, must understand the three dimensional anatomy of the spine, and must carefully select appropriate patients for surgery. The pedicle is the strongest portion of the vertebrae, transmitting all forces from the posterior elements to the vertebral body. It can withstand stresses of rotation, side bending, and extension of the spine, and is an ideal structure to lock into and control with posterior instrumentation when spinal fixation is needed.

Pedicle screw fixation rigidly immobilizes all three columns of the spine while requiring only the presence of an intact pedicle. While inherent advantages and disadvantages are found in each pedicle screw system made, specific indications for use do not vary. Successful use of each system requires appropriate preoperative planning, an appreciation of spinal biomechanics, and correct application of the device.

The principal indications for pedicle screw fixation include conditions of translational instability (spondylolisthesis, degenerative spondylolisthesis, and fracture), conditions of axial instability (tumor, fracture, degenerative disease), conditions of mechanical pain (pseudarthrosis, discogenic back pain, and adjacent level instability), and conditions of deformity (scoliosis, degenerative scoliosis, flat-back syndrome, and spondyloarthropathies).

Trauma of the thoracolumbar spine and lumbosacral spine is an important indication for pedicular fixation: fewer functional spinal units need to be instrumented when compared with nonpedicular spinal fixation devices, preserving normal lumbar mechanics and motion. Another indication is acquired instability or hypermobility following decompressive spinal surgery in patients with spinal stenosis. When one entire facet or half of both facets have been removed, some authors suggest that a spinal fusion is necessary to prevent spondylolisthesis. Lumbosacral fixation, a challenge in many spinal deformity patients, can be greatly enhanced with transpedicular fixation devices.

Pedicle screws have Food and Drug Administration approval for use in the thoracic, lumbar, and sacral spine, to provide immobilization and stabilization of spinal segments in skeletally mature patients. Pedicle screws are currently approved by the FDA for use in acute and chronic instabilities and deformities of the spine, degenerative spondylolisthesis, fracture, dislocation, scoliosis, kyphosis, tumor, and pseudarthrosis of the spine. Pedicle screws are considered a Class III device for all other applications.

Contraindications to pedicle screw instrumentation include severe osteopenia or osteoperosis, inadequate pedicle dimensions, congenital or absent pedicles, fractures involving the pedicle, and active infection. Poor mechanical purchase within a damaged pedicle is a relative contraindication.

A unique probe design is proposed herein to allow transpedicular aspiration of marrow elements including osteoprogenitor cells used in spine fusion augmentation. The probe will simultaneously create a pilot hole for pedicle screw instrumentation and provide access to the rich marrow beds contained within the vertebral bodies.

The probe system will allow surgeons to speed the process of surgical stabilization without sacrificing efficacy of spinal fusion. The procedure will eliminate other graft harvesting techniques which carry a measurable incidence of complications and morbidity, without adding to the risk or time of the traditional pedicle screw placement operation.

### SUMMARY OF THE INVENTION

In accordance with the present invention, a method for aspirating bone marrow through a human vertebra is provided. The method comprises the steps of forming an opening in a bone in a patient's body, aspirating marrow from the bone through the opening in the bone, and positioning a screw in the opening through which marrow was aspirated.

In accordance with one exemplary embodiment of the present invention, an apparatus and method for use in aspirating bone marrow is provided. The apparatus comprises a tube which has a body with a distal end portion. The tube has a central opening at one end of a central passage extending through the tube and through which bone marrow is aspirated. The body of the tube has a tapered shaft. At least one aspiration opening at the distal end of the tube is used to further aspirate bone marrow.

In accordance with another exemplary embodiment of the present invention, a probe for use in aspirating bone marrow is provided. The probe comprises a hollow tube having a proximal end portion, a distal end portion, and a central passage extending therebetween through which bone marrow is aspirated. The distal end portion comprises a shovel-shaped tip and has a central opening at one end of the central passage. The tube further comprises a tapered shaft extending between the proximal end portion and the distal end portion. At least one aspiration opening is disposed at the distal end of the tube, and extends from an outer surface of the tube to the central passage. An inner trochar which has a proximal end and a distal end is also provided. The proximal end includes a trochar thread convolution which engages a tube thread convolution on the tube to interconnect the trochar and the tube. The distal end of the trochar includes four sides which intersect at a point to form a sharp end for penetrating bone. The sharp end is disposed distal to the shovel-shaped tip when the inner trochar and the tube are interconnected.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1A is a front view of a tube in accordance with the present invention;

FIG. 1B is a side view of the tube of FIG. 1;

FIG. 2 is a top view of the tube of FIG. 1;

FIG. 3A is a front view of an inner trochar in accordance with the present invention;

FIG. 3B is a side view of the inner trochar of FIG. 3A;

FIG. 4A is a front view of an assembled apparatus for aspirating bone marrow in accordance with the present invention;

FIG. 4B is a side view of the apparatus of FIG. 4A;

FIG. 5 is a schematic illustration of the human spinal column;

FIG. 6 is a schematic illustration of the apparatus of FIG. 4 being inserted into a vertebra of a spine of a human being;

FIG. 7 is an enlarged view of the insertion of FIG. 6;

FIG. 8A is an enlarged top view of the distal end of the tube of FIG. 7;

FIG. 8B is an enlarged top view of the distal end of a tube in accordance with an alternative construction of the present invention;

FIG. 9 is a sectional view taken along lines 9-9 in FIG. 7 of the distal end of the apparatus;

FIG. 10 is a sectional view of the apparatus inserted into the pedicle of the vertebra;

FIG. 11 is a sectional view of the apparatus inserted into the vertebral body of the vertebra;

FIG. 12 is a sectional view of the apparatus aspirating bone marrow from the vertebral body;

FIG. 13 is a section view of a pedicle screw placed in a shaft remaining in the vertebra after removal of the apparatus from the vertebra;

FIG. 14 is a side view of a tube in accordance with a second embodiment of the present invention;

FIG. 15 is a bottom view of the tube of FIG. 13 taken along lines 15-15 in FIG. 14;

FIG. 16 is a side view of an inner trochar in accordance with a second embodiment the present invention;

FIG. 17A is an enlarged top view of the distal end of the trochar of FIG. 16;

FIG. 17B is an enlarged side view of the distal end of the trochar of FIG. 16;

FIG. 18 is a side view of an apparatus for aspirating bone marrow in accordance with a second embodiment of the present invention;

FIG. 19A is a side view of an adapter used to aspirate bone marrow in accordance with the second embodiment of the present invention; and

FIG. 19B is a side view of another adapter used to aspirate bone marrow in accordance with the second embodiment of the present invention.

### DETAILED DESCRIPTION

The present invention relates to a probe 80 that is used to aspirate bone marrow 104 from a human bone, and in particular, the marrow progenitor cells from the cancellous bone of the vertebral body 92. A shaft 100 in the bone left by the probe 80 is then utilized for pedicle screw placement.

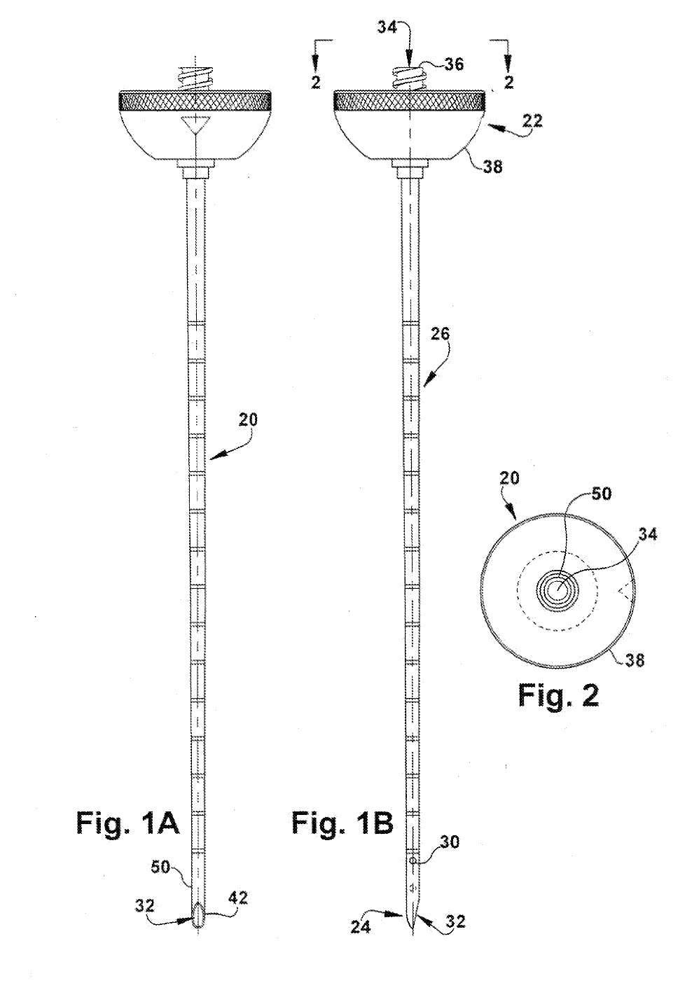

The probe 80 comprises an inner trochar 60 that is inserted into, and engageable with, a hollow tube 20. As shown in FIGS. 1-2, the hollow tube 20 comprises a proximal end portion 22 and a distal end portion 24 with a body portion 26 extending therebetween. The tube 20 is generally cylindrical in nature and includes a central passage 34 extending the entire length of the tube 20. Although the cross section of the tube 20 is depicted as being circular in nature, it is understood that the tube 20 may be rectangular, triangular, or the like. Additionally, the diameter or cross-section of the body portion 26 of the tube 20 tapers slightly inwardly from the proximal end 22 to the distal end 24. This taper resists dangerous plunging of the tube tip, which can occur with any tube 20 advancement under direct pressure. The tube 20 is made of a suitable biocompatible metal or polymer, and is generally strong enough to be advanced through the vertebra 90 without plastic deformation.

The proximal end 22 of the tube 20 includes a handle 38 and an external thread convolution 36. The handle 38 has a larger footprint than the tube 20 and provides the user with a larger surface area with which to grasp and articulate the tube 20, as will be hereinafter described. The handle 38 is hemispherical in nature or otherwise suitable for grasping. The threads 36 on the proximal end of the tube 20 have a Luer-Lock configuration and are designed to threadably engage an internal thread convolution 72 on the inner trochar 60 once the inner trochar 60 is disposed within the central passage 34 of the tube 20.

The distal end 24 of the tube 20 comprises a shovel-shaped tip 42 that allows the user to dissect and auger through trabecular bone within the pedicle 94 and vertebral body 92 while avoiding inadvertent penetration of the cortical walls of the pedicle 94. The nature of the shovel-shaped tip 42 will be discussed in greater detail hereinafter. The distal end 24 further includes a central opening 32 disposed at the distal end of the central passage 34. The central opening 32 is sized to receive the inner trochar 60.

At least one aspiration opening 30 is disposed in proximity with the tip 42 of the tube 20. The openings 30 are disposed around the periphery of the tube 20 and extend from an outer surface 50 of the tube 20 to the central passage 34. Although four openings 30 are depicted in the phantom lines of FIG. 2, any number of openings 30 may be used, including one. The openings 30 serve as a means by which bone marrow 104 can be circumferentially aspirated from a site within the bone, through the central passage 34, and out the proximal end 22 of the hollow tube 20 to a collection reservoir. The openings 30 may be circular or elliptical in nature, or otherwise suitable to allow bone marrow 104 to pass from the outer surface 50 of the tube 20 into the central passage 34.

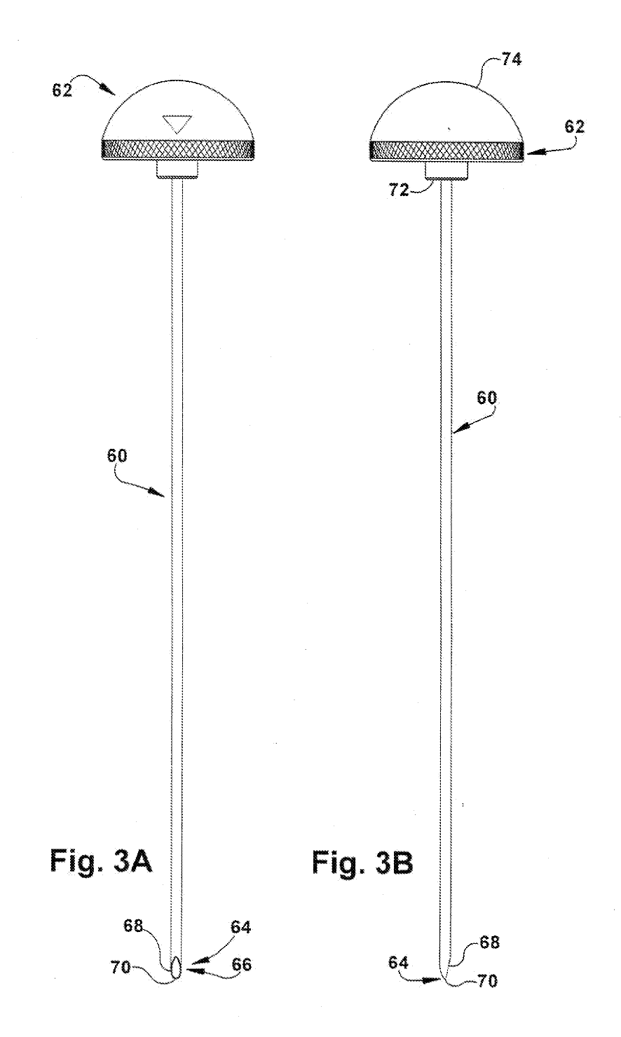

The inner trochar 60 is depicted in FIGS. 3A-3B. The inner trochar 60 is generally cylindrical in nature and comprises a proximal end 62 and a distal end 64. The trochar 60 is made of a suitable biocompatible metal or polymer, and is generally made of a more durable and harder material than the tube 20. The profiles of the trochar 60 and tube 20 are such that, when the trochar 60 is disposed within the tube 20, the trochar 60 and tube 20 are substantially concentric. The proximal end 62 of the trochar 60 includes a knurled knob 74 having an internal thread convolution 72 underneath its distal end that engages with the external thread convolution 36 on the proximal end 22 of the hollow tube 20. The knob 74 is hemispherical in nature or otherwise shaped to allow the user to easily engage and disengage the threads 72 of the inner trochar 60 with the threads 36 of the tube 20.

The distal end 64 of the trochar 60 comprises a sharp, four-point starter tip 66 that is used to breach the cortex 96 of the bone. The tip 66 is comprised of four sides 68 that intersect at a point to form a sharp end 70 of the trochar 60 for penetrating bone. The portion of the tube 20 proximal to the four sides 68 of the tip 66 is formed to sit flush with the shovel-shaped tip 42 of the hollow tube 20 once the probe 80 is assembled.

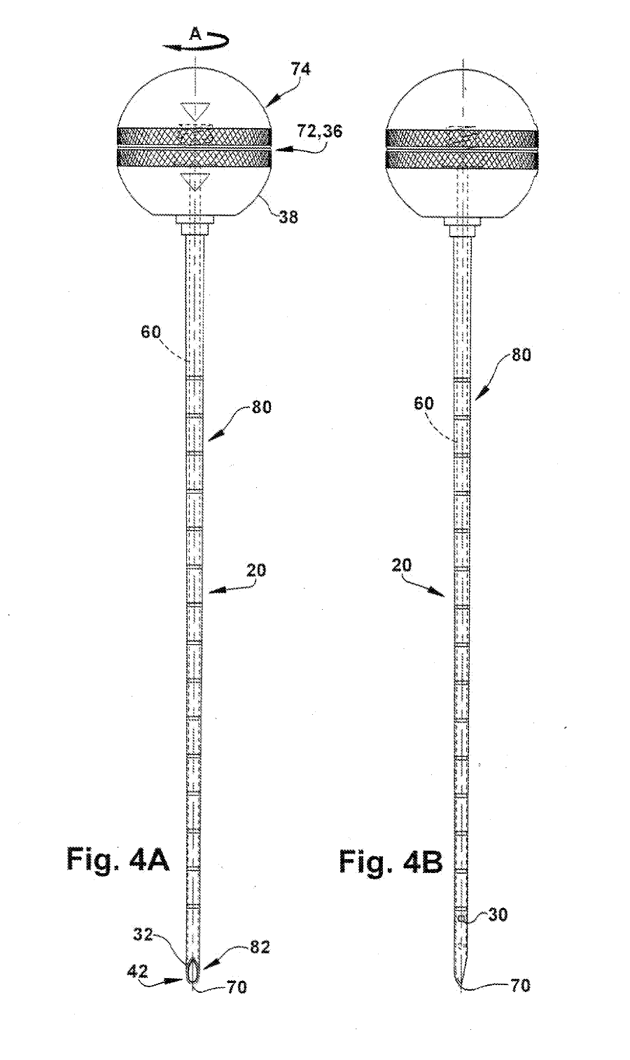

The assembled probe 80 is shown in FIGS. 4A-4B. To assemble the probe 80, the distal end 64 of the trochar 60 is inserted into the central passage 34 of the hollow tube 20 at the proximal end 22 and fed towards the distal end 24 of the tube 20 until the sharp end 70 of the trochar 60 aligns with the central opening 32 of the tube 20 and past the shovel-shaped tip 42 of the tube 20. Thus, the sharp end 70 of the trochar 60 combines with the shovel-shaped tip 42 to form the leading edge of the probe 80 as it is inserted or advanced into bone. The trochar 60 is locked in place relative to the tube 20 by engaging the threads 72 of the trochar 60 with the threads 36 of the tube 20. Clockwise rotation A of the knob 74 on the trochar 60 will tighten the threads together and fully assemble the probe 80 to this configuration. In the alternative, the assembly eschues the threads 72 to allow the inner trochar 60 to slide directly through the tube 20.

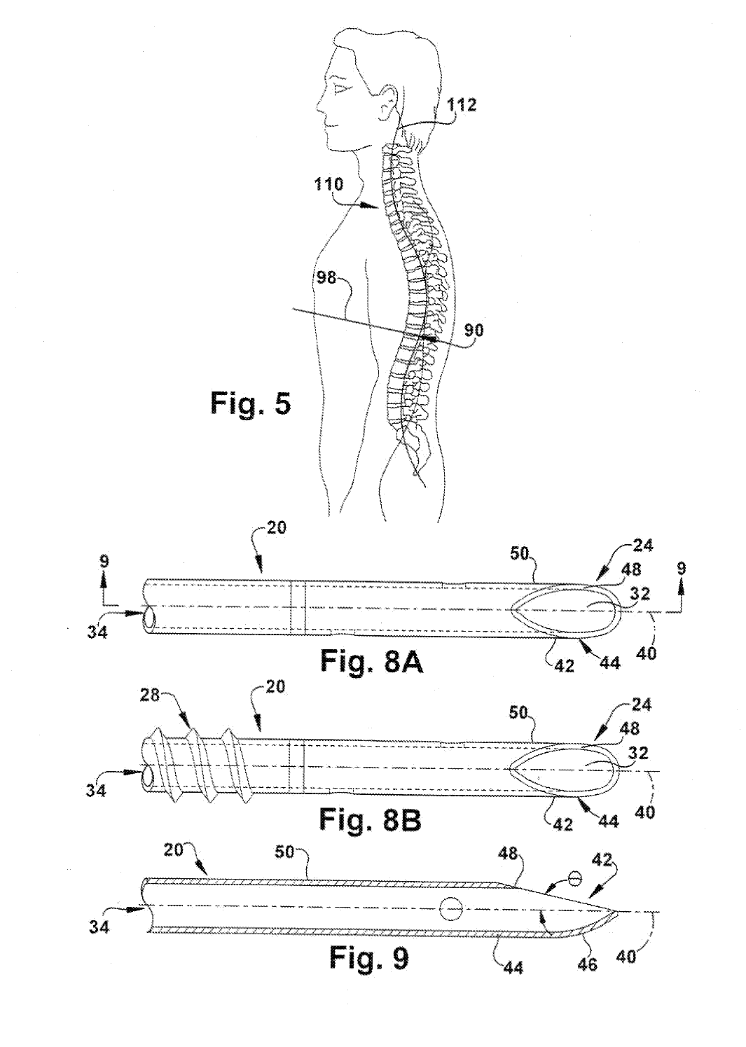

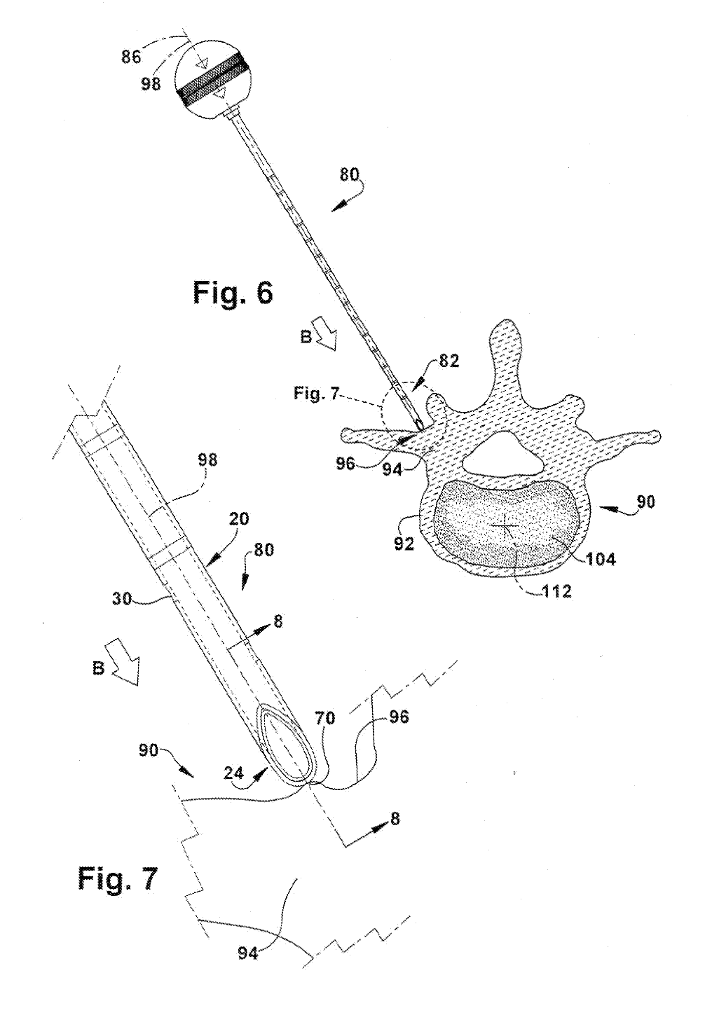

In operation, the probe 80 is inserted and driven into a vertebra 90 of the patient in order to aspirate bone marrow 104 from the vertebral body 92. As shown in FIGS. 5 and 6, the distal end 82 of the probe 80 is placed in contact with the pedicle 94 on a vertebra 90 of the spine 110. The probe 80 is oriented such that a longitudinal central axis 86 of the probe 80 is co-axial with an insertion axis 98. The insertion axis 98 extends through the pedicle 94 of the vertebra 90 and into the vertebral body 92 where bone marrow 104 is located. This insertion axis 98 is orthogonal to, and offset from, an axis 112 running the length of the patient's spinal column along the contour of the spine 110. Maintaining an aspiration hole orthogonal to the spine and coaxial with the pedicle 94 is critical for likewise creating orthogonal pedicle screw placement pilot holes to facilitate spinal instrumentation fixation. Although a vertebra 90 of the lumbar region of the spine 110 is depicted, it is understood that the probe 80 may be inserted into any vertebra (i.e. cervical, thoracic, or lumbar) for the placement of pedicle screws for spinal instrumentation fixation.

As shown in FIG. 7, the configuration of the probe 80 is such that the sharp end 70 of the inner trochar 60 acts as the leading edge for the probe 80. The sharp end 70 is driven in the direction B along the insertion axis 98 to breach the vertebral cortex 96 at the pedicle 94 of the vertebra 90. Breaching of the cortex 96 with an awl before inserting the end 70 of the inner trochar 60 is necessary because the tip 42 of the tube 20 is too blunt to puncture the cortex. This breaching will allow subsequent insertion of the probe 80 within the pedicle 94 and ultimately the vertebral body 92 for the harvesting of marrow 104.

FIGS. 8A and 9 depict detailed drawings of the shovel-shaped tip 42 at the time of pedicular breach. The shovel-shaped tip 42 can generally be described as a hollow cylindrical tube 20 with a closed end that is subsequently machined at an angle Θ to create an opening with a scooped tip. The hollow tube 20 generally comes to a rounded or blunt tip at its distal end 24 (FIG. 8). As noted, the nature of the tip 42 allows the user to drive through cancellous bone while avoiding inadvertent penetration of the cortical walls of the pedicle 94. The tip 42 comprises a continuation of a cylindrical side wall 44 of the central passage 34 that slopes along an arcuate path towards a longitudinal centerline 40 of the tube 20 to form a concave surface 46 (FIG. 9). The concave surface 46 partially blocks the distal end portion of the central passage 34. Although the concave surface 46 is shown in FIG. 9 to extend above the centerline 40 of the tube 20, it will be appreciated that the concave surface 46 could likewise terminate below or at the centerline 40.

FIG. 8B depicts an alternative construction of the present invention in which the distal end 24 of the body portion 26 of the tube 20 includes an external thread convolution 28. The thread convolution 28 is oriented along the distal end 24 of the body portion 26 of the tube 20 such that at least one aspiration opening 30 is disposed between the thread convolution 28 and the tip 42 of the tube 20. The thread convolution 28 may be designed to engage the bone and secure the probe 80 while bone marrow 104 is aspirated, and to control and facilitate advancement of the probe 80 through the cancellous bone. As will be hereinafter described, the thread convolution 28 creates a threaded pilot hole 100 in which a pedicle screw 114 is placed once the aspiration has been completed and the probe 80 removed from the bone. Alternatively, the thread convolution 28 may create a smooth pilot hole 100 which is subsequently tapped to receive pedicle screw 114.

Regardless, the shovel-shaped tip 42 further comprises the central opening 32, which is also defined by the side wall 44 of the central passage 34 (FIG. 9). At the tip 42 of the tube 20, the side wall 44 is machined or otherwise formed at an angle Θ relative to the centerline 40 to create a sloped portion 48. It is this sloped portion 48 of the side wall 44 that defines the central opening 32. The central opening 32 is depicted as being oblong in nature, but may also be circular, triangular, or the like, depending on the nature of the sloped portion 48. The sloped portion 48 intersects the concave surface 46 to form the shovel-shaped tip 42.

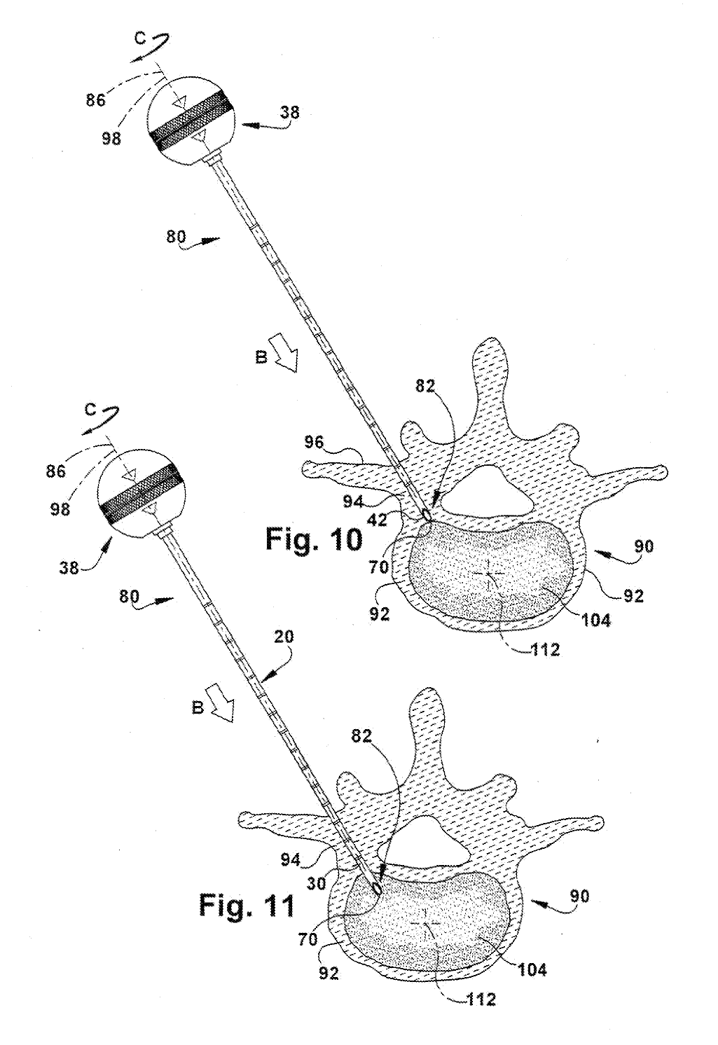

Once the harder cortex 96 of the pedicle 94 has been breached, advancement of the distal end 82 of the probe 80 through the cancellous bone of the pedicle 94 is facilitated by the shovel-shaped tip 42 of the tube 20. As shown in FIG. 10, the distal end 82 of the probe 80 is advanced through the cancellous bone of the pedicle 94 by a combination of a reciprocating rotational movement C of the handle 38 and a force in the axial direction B on the probe 80 along the insertion axis 98 towards the vertebral body 92. Such movement allows the shovel-tip 42 of the tube 20 and the sharp end 70 of the inner trochar 60 to drive through the cancellous bone of the pedicle 94.

Where the probe 80 includes the external thread convolution 28 along the body portion 26 of the tube 20, the probe 80 is advanced in this fashion until the external thread convolution 28 of the tube 20 begins to engage the cortex of the pedicle 94 (not shown). Once the external thread convolution 28 of the tube 20 is engaged with the cortex of the pedicle 94, further advancement of the probe 80 into the vertebra 90 is achieved by rotating the handle 38 of the tube 20 clockwise to further engage the external thread convolution 28 of the tube 20 into the cancellous bone of the pedicle 94. This rotation screws the probe 80 into the pedicle 94 and advances the probe 80 along the insertion axis 98 towards the vertebral body 92 and its bone marrow 104.

Whether the probe 80 includes the external thread convolution 28 or not, advancement into the vertebra 90 creates a shaft 100 in the bone along the insertion axis 98. As noted, this shaft 100 may be tapered if the probe 80 includes the external thread convolution 28. Continued rotation in this fashion in the direction C subsequently drives the distal end 82 of the probe 80, and thus the distal end 24 of the tube 20, into the marrow 104 of the vertebral body 92 at the anterior side of the vertebra 90 (FIG. 11). The advancement of the probe 80 in this fashion simultaneously creates an internal pilot hole 102 in the shaft 100 through the pedicle 94 along the insertion axis 98. It is this internal pilot hole 102 in the shaft 100 that will subsequently receive a pedicle screw 114 for spinal implant fixation.

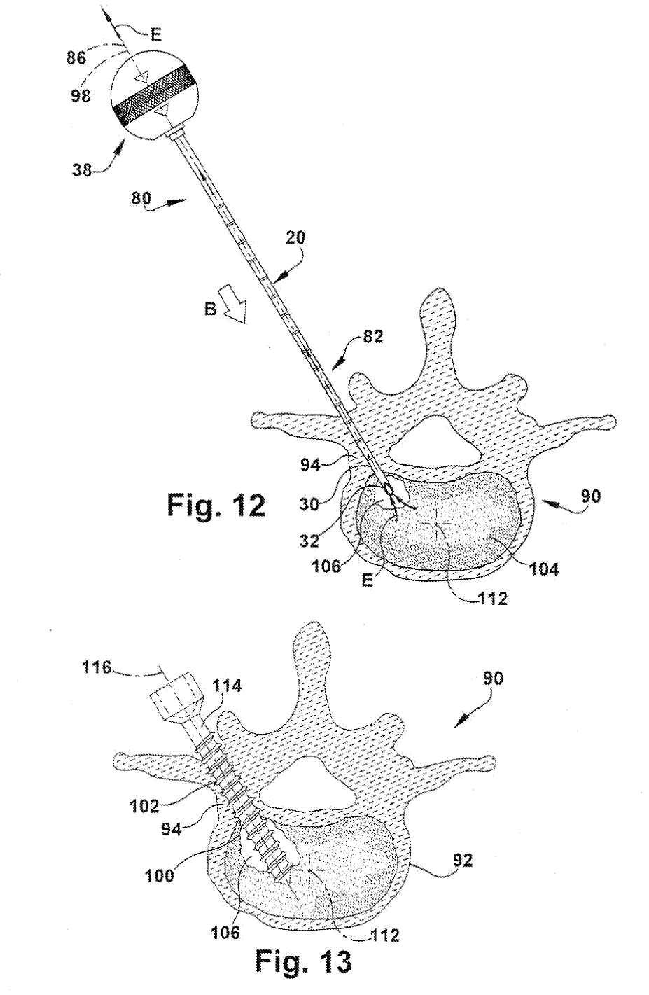

The probe 80 is advanced until the opening 30 in the distal end 24 of the tube 20 is disposed within the bone marrow 104 of the vertebral body 92 as shown in FIG. 12. At this time, the inner trochar 60 is unthreaded from the proximal end 22 of the tube 20 and removed from the central passage 34 (FIG. 12). Removal of the inner trochar 60 renders the proximal end 22 of the tube 20 in fluid communication with the aspiration opening 30 and central opening 32 at the distal end 24 of the tube 20 via the central passage 34. Furthermore, as the shaft of the tube 20 is tapered and compresses the cancellous bone of the pedicle 94, the tube 20 also seals the shaft 100 in the bone created by the driven probe 80 and prevents air and peripheral blood from being drawn into the tip 42 of the tube. Such air and blood presence at the tip 42 of the tube 20 would dilute the bone marrow 104 cells as they are harvested through the probe 80.

With the tube 20 fixed within the pedicle 94 of the vertebra 90, aspiration of bone marrow 104 can begin. The aspiration may be carried out using the Cellect™ system offered by DePuy of Raynham, Mass., but any known method of aspiration may alternatively be used. Suction means (not shown) in fluid communication with the proximal end 22 of the tube 20 apply suction to the central passage 34 of the tube 20 and pull bone marrow 104 into the central opening 32 and aspiration openings 30 at the distal end 24 of the tube. As indicated by arrow E (FIG. 12), the bone marrow 104 is drawn into the distal end of the central passage 34, aspirates through the body 26 of the tube 20, and subsequently is pulled out through the proximal end 22 of the tube 20 as indicated by arrow F and into a reservoir or other storage container for preservation and collection. The orientation of the aspiration openings 30 allow peripheral aspiration of bone marrow 104 around the entire circumference of the distal end 24 of the tube 20. Thus, bone marrow 104 is aspirated around the vicinity of the distal end 24 of the tube 20 such that a larger void 106 than merely the contour of the tube 20 is created in the vertebral body 92. That is, a relatively large amount of bone marrow 104 is capable of being aspirated while the tube 20 is disposed along a single line within the vertebra 90 without movement or angulation of the tip 42 of the tube 20.

Once all the bone marrow 104 has been collected from the first designated depth into the vertebral body 92, the process is repeated. The inner trochar 60 is re-inserted into the central passage 34 of the tube 20 and then threadably engaged with the tube 20 such that the sharp end 70 of the inner trochar 60 is disposed distal to the shovel-shaped tip 42 of the tube 20. Reinsertion of the trochar 60 simultaneously clears bone and other debris from the central passage 34 of the tube 20 any time the central passage and/or tip 42 of the tube 20 becomes clogged. This clearing of the central passage 34 will facilitate subsequent aspiration of bone marrow 104 at greater depths in the vertebral body 92.

The probe 80 is then both rotated in the clockwise direction C and forced in the axial direction B to advance the distal end 82 of the probe 80 along the insertion axis 98 farther into the vertebral body 92 of the vertebra 90. Generally, after initial marrow aspiration, the probe 80 is advanced approximately 5.0-10.0 mm in between aspiration cycles. Once the desired position is reached along the insertion axis 98, the inner trochar 60 is again removed, and aspiration of the bone marrow 104 surrounding the new position of the tip 42 of the tube 20 is carried out. This process is repeated until the desired volume of bone marrow 104 has been aspirated from the vertebral body 92 (FIG. 13).

Alternatively, the tube 20 can be placed within the vertebra 90 via guided placement. Under fluoroscopic control, a guidewire and introducer (not shown) are advanced through the laminar cortex 96 and coaxially down the pedicle 94, stopping in the vertebral body 92. The guidewire is advanced slightly as the introducer is removed. The tube 20 is then passed over the guidewire down to the laminar entry point, and then driven into the vertebra 90 up to the external thread convolution 28. After advancing the tube 20 to the proper depth the guidewire is removed and repeated aspiration carried out as above.

Utilizing either direct or guided placement, following the final aspiration, the tube 20 is removed from the vertebra 90 by rotating the handle 38 in the counterclockwise direction. FIG. 13 depicts the vertebra 90 with the tube 20 removed and a large aspiration void 106 in the bone marrow 104 of the vertebral body 92 where bone marrow 104 was aspirated from. A pedicle screw 114 is threaded into the shaft 100 left from the insertion of tube 20 by engaging the screw 114 with the internal pilot hole 102 of the shaft 100. The screw 114 has a longitudinal central axis 86 which is co-axial with the insertion axis 98 as it is threaded into the pedicle 94. Once in place, the axis 116 of the screw 114 is orthogonal to, and offset from, the spine axis 112 running the length of the patient's spine 110 (FIGS. 5 and 6). The screw 114 can be used to fixate any spinal implantation device or the like to the spine 110. The probe 80 may be used to create similar holes in the contralateral pedicle 94 of the same vertebra 90 and/or on the pedicles of different vertebra along the spine 110. This allows for accurate placement of multiple pedicle screws 114 along a patient's spine 110 to accommodate a range of spinal fixation instrumentation.

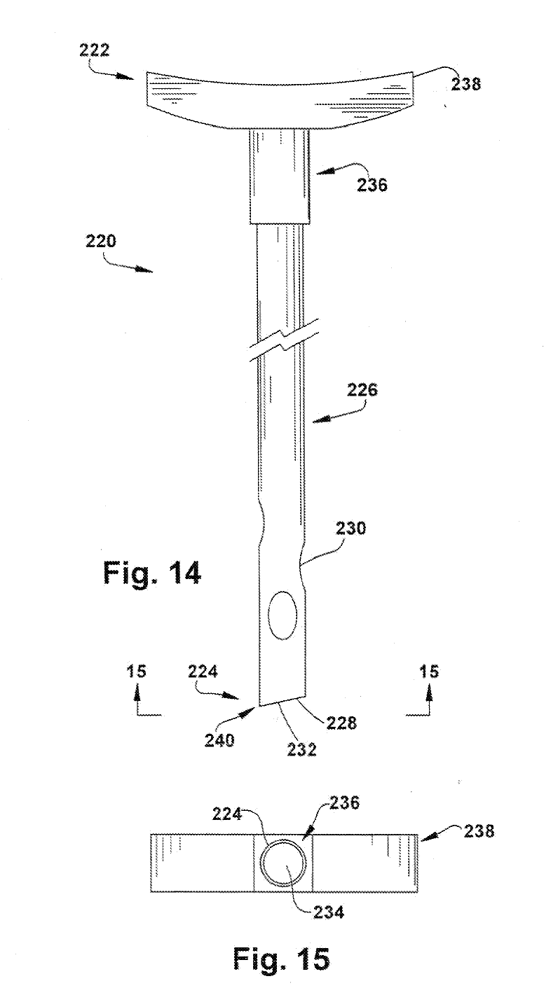

In an alternative embodiment of the present invention, the probe 280 comprises an inner trochar 260 that is inserted into a hollow tube 220. As shown in FIGS. 14-15, the hollow tube 220 comprises a proximal end portion 222 and a distal end portion 224 with a body portion 226 extending therebetween. The tube 220 is generally cylindrical in nature and includes a central passage 234 extending the entire length of the tube 220. Although the cross section of the tube 220 is depicted as being circular in nature, it is understood that the tube 220 may be rectangular, triangular, or the like. Additionally, the diameter or cross-section of the body portion 226 of the tube 220 may taper slightly inwardly from the proximal end 222 to the distal end 224. This taper resists dangerous plunging of the tube tip, which can occur with any tube 220 advancement under direct pressure. The tube 220 is made of a suitable biocompatible metal or polymer, and is generally strong enough to be advanced through the vertebra 90 without plastic deformation.

The proximal end 222 of the tube 220 includes a handle 236 having a flange portion 238. The handle 236 has a larger footprint than the tube 220 and provides the user with a larger surface area with which to grasp and articulate the tube 220. The handle 236 has a T-shape, but may also have another shape suitable for grasping.

The distal end 224 of the tube 220 comprises a tapered tip 228 and a central opening 232 disposed at the distal end of the central passage 234. The central opening 232 is sized to receive the inner trochar 260.

At least one aspiration opening 230 is disposed proximal to the tip 228 of the tube 220. The openings 230 are disposed around the periphery of the tube 220 and extend from an outer surface 242 of the tube 220 to the central passage 234. Although three openings 230 are depicted in the phantom lines of FIG. 15 radially spaced apart 1200, any number of openings 230 may be used, including one, in any radial configuration. Furthermore, openings 230 are depicted as being longitudinally spaced apart. However, openings 230 may likewise lie in substantially the same longitudinal plane. The openings 230 serve as a means by which bone marrow 104 can be circumferentially aspirated from a site within the bone, through the central passage 234, and out the proximal end 222 of the hollow tube 220 to a collection reservoir. The openings 230 may be circular or elliptical in nature, or otherwise suitable to allow bone marrow 104 to pass from the outer surface 242 of the tube 220 into the central passage 234.

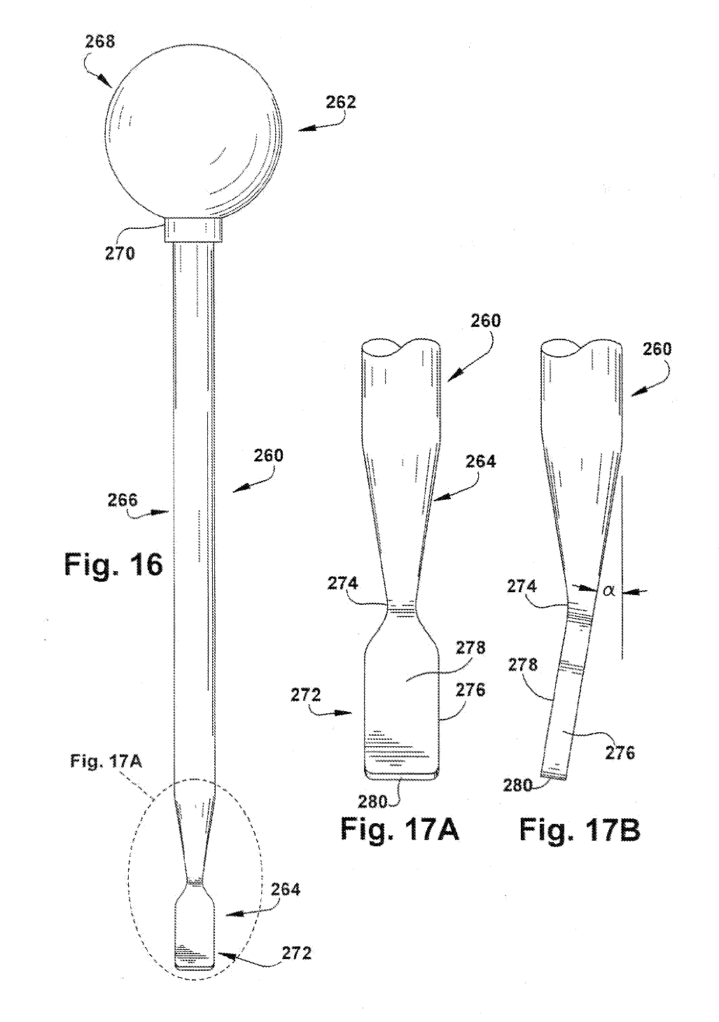

The inner trochar 260 is depicted in FIG. 16. The inner trochar 260 is generally tubular in nature and comprises a proximal end 262 and a distal end 264. The trochar 260 is made of a suitable biocompatible metal or polymer, and is generally made of a more durable and harder material than the tube 220. The profiles of the trochar 260 and tube 220 are such that, when the trochar 260 is disposed within the tube 220, the trochar 260 and tube 220 are substantially concentric. The proximal end 262 of the trochar 260 includes a round handle 268 having a projection 270 extending therefrom. The handle 268 is preferably phenolic, but may be made of any suitable plastic or polymer. The projection 270 is configured to releasably engage the flange 238 of the tube 220. The projection 270 is rectangular in nature, or otherwise shaped to allow the user to easily engage and disengage the projection 270 with the flange 238 of the tube 220.

As shown in FIGS. 17A-B, the distal end 264 of the trochar 260 comprises a tip 272 having a rounded end 280 for blunt sounding of the cortex 96 of the pedicle 94. The distal end 264 tapers radially inwardly to form a neck portion 274 which transitions into shovel-shaped portion 276. The shovel 276 comprises an outer surface 278, and has a slight curve at angle α to permit manipulation of the probe 290 within the pedicle 94. When probe 290 is assembled, tapered tip 228 of tube 220 will lie adjacent to neck portion 274 of the trochar 260.

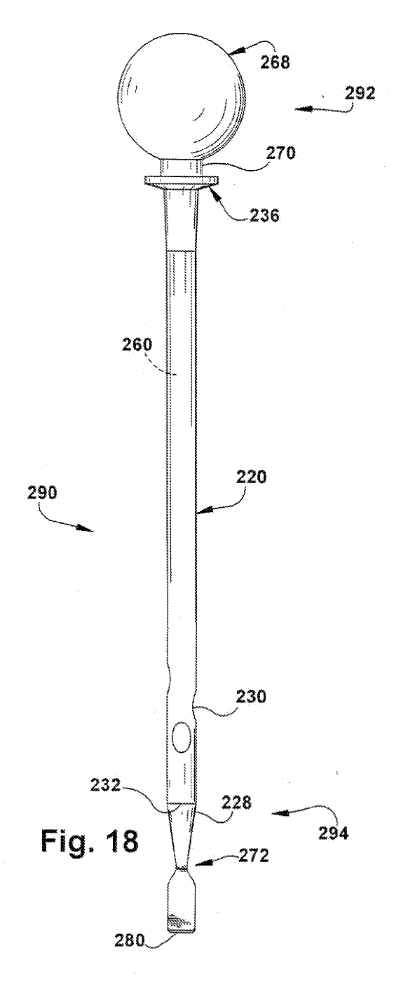

The assembled probe 290 is shown in FIG. 18. To assemble the probe 290, the distal end 264 of the trochar 260 is inserted into the central passage 234 of the hollow tube 220 at the proximal end 222 and fed towards the distal end 224 of the tube 220 until the rounded end 280 and shovel 276 of the trochar 280 protrude out from the central opening 232 of the tube 220 and past the tip 228 of the tube 220. Thus, the rounded end 280 of the trochar 260 is the leading edge of the probe 290 as it is bluntly sounded into bone. The trochar 260 is locked in place relative to the tube 220 by engaging the projection 270 of the trochar 260 with the flange 238 of the tube 220.

The operation of probe 290 is substantially similar to that of probe 80. However, in this alternative embodiment, the cortex 96 of pedicle 94 is breached by the blunt sounding of rounded end 280 of trochar 260 of the assembled probe 290. It will be appreciated that inner trochar 60 with sharp end 70, instead of inner trochar 260 with rounded end 280, could be used with inner tube 220 to breach the cortex 96 of pedicle 94. Furthermore, because tube 220 does not include the external thread convolution 28 of tube 20, once the distal end 294 of probe 290 has breached the cortex 96 of pedicle 94, the probe 280 is advanced through the vertebra 90 by additional blunt sounding, and not by clockwise rotation of the probe.

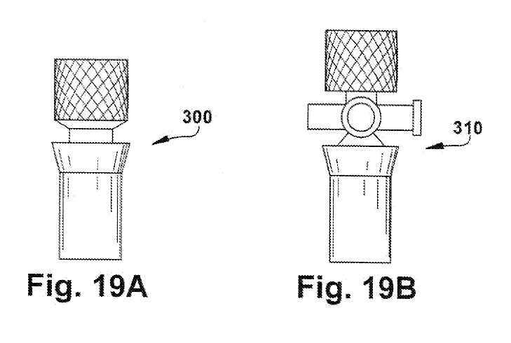

The sequential aspiration and advancement of probe 280 is also substantially similar to that of probe 80. The angled orientation of shovel 276 helps probe 290 auger through the trabecular bone towards the vertebral body 92. When each desired aspiration location is reached within the vertebra 90, the trochar 260 is removed to allow adapter 300 or three-way adapter 310 (FIGS. 19A-B) to connect to the flange 238 of inner tube 220. This connection will allow any Luer-Lock type syringe to attach to the adapter 300, 310 for aspiration. The process is repeated until all desired aspiration has been completed. Subsequently, as with probe 20, probe 290 is removed from vertebra 90 and shaft 100 is adapted to accept pedicle screw 114. The entire process is repeated until the all required pedicle screws 114 are implanted along spine 110.

From the above description of the invention, those skilled in the art will perceive improvements, changes and modifications. Such improvements, changes and modifications within the skill of the art are intended to be covered by the appended claims.

Having described the invention, the following is claimed:

1. A method comprising the steps of:
forming an opening in a bone in a patient's body;
aspirating marrow from the bone through the opening in the bone; and
positioning a screw in the opening through which marrow was aspirated.

2. The method of claim 1 wherein the step of forming the opening in the bone in the patient's body comprises the step of forming an opening in a vertebrae.

3. The method of claim 2 wherein the step of positioning a screw in the opening through which marrow was aspirated includes threading the screw into the opening with the screw offset to one side of a longitudinal central axis of a spine and with a central axis of the screw transverse to the longitudinal central axis of the spine.

4. The method of claim 1 wherein the step of forming an opening in a bone in a patient's body includes engaging a tapered shaft on a hollow tube with the bone.

5. The method of claim 4 wherein the step of forming an opening in a bone in a patient's body further includes aspirating marrow from the bone through at least one opening in the hollow tube.

6. An apparatus for use in aspirating bone marrow, said apparatus comprising:
a tube having a body with a distal end portion, said tube having a central opening at one end of a central passage extending through the tube and through which bone marrow is aspirated, said body further having a tapered shaft; and
at least one aspiration opening disposed at said distal end portion of said tube.

7. The apparatus of claim 6, wherein said distal end portion of said tube has a tip comprising an arcuate concave surface which forms a continuation of a cylindrical side wall of said central passage, said concave surface slopes toward a longitudinal centerline of said hollow tube and partially blocks said central passage, said tip further comprises said central opening, which is defined by said side wall and having a sloped portion which intersects said concave surface, and is formed at an angle relative to said centerline.

8. The apparatus of claim 6 further comprising a thread convolution on said distal end portion of said body.

9. The apparatus of claim 7 further comprising an inner trochar having a proximal end and a distal end, said distal end including a tip congruent with the tube for penetrating bone, said proximal end having a thread convolution which engages a tube thread convolution on said tube to interconnect said trochar and said tube.

10. A probe for use in aspirating bone marrow, said probe comprising:
a hollow tube having a proximal end portion, a distal end portion, and a central passage extending therebetween through which bone marrow is aspirated, said distal end portion comprising a shovel-shaped tip and having a central opening at one end of said central passage, said tube further comprising a tapered shaft extending between said proximal end portion and said distal end portion, wherein at least one aspiration opening is disposed at said distal end portion of said tube, and extends from an outer surface of said tube to said central passage; and
an inner trochar having a proximal end and a distal end, said proximal end including a trochar thread convolution which engages a tube thread convolution on said tube to interconnect said trochar and said tube, said distal end of said trochar including four sides which intersect at a point to form a sharp end for penetrating bone, wherein said sharp end is disposed distal to said shovel-shaped tip of said tube when said inner trochar and said tube are interconnected.

11. The apparatus of claim 10, wherein said tapered shaft of said tube includes a thread convolution at said distal end portion of said tube.

12. The apparatus of claim 10, wherein said distal end of said trochar is shovel-shaped.

13. The apparatus of claim 10, wherein said shovel-shaped tip comprises an arcuate concave surface which forms a continuation of a cylindrical side wall of said central passage, said concave surface slopes toward a longitudinal centerline of said hollow tube and partially blocks said central passage, said tip further comprising said central opening, which is defined by said side wall and having a sloped portion which intersects said concave surface, and is formed at an angle relative to said centerline.

14. The apparatus of claim 10, wherein said tube further comprises a handle at said proximal end portion of said tube, wherein the diameter of said tube decreases from said proximal end of said tube to said distal end of said tube.
